(19) United States
(12) Patent Application Publication
(10) Pub. No.: US 2023/0193172 A1
(43) Pub. Date: Jun. 22, 2023

(54) ENZYME PRODUCTION USING ONE OR MORE CO-PRODUCT COMPOSITIONS FROM A BIOPROCESSING FACILITY

(71) Applicant: POET Research, Inc., Sioux Falls, SD (US)

(72) Inventors: Cory James Sarks, Sioux Falls, SD (US); Benjamin P. Gacke, Baltic, SD (US); Stephen M. Lewis, Sioux Falls, SD (US); Jennifer Rebecca Headman, Sioux Falls, SD (US)

(21) Appl. No.: 18/086,350

(22) Filed: Dec. 21, 2022

Related U.S. Application Data
(60) Provisional application No. 63/292,730, filed on Dec. 22, 2021.
(60) Provisional application No. 63/338,184, filed on May 4, 2022.

(51) Int. Cl. C12F 3/10

(52) U.S. Cl. CPC C12F 3/10

(57) ABSTRACT

Enzyme production in a bioprocessing facility that utilizes the enzyme that is produced. Enzyme is produced by one or more microorganisms grown on a co-product (e.g., one or more stillage compositions) of the bioprocessing facility.

### CROSS-REFERENCE TO RELATED APPLICATIONS

The present nonprovisional application claims the benefit of commonly owned provisional applications: Ser. No. 63/292,730, filed on Dec. 22, 2021; and Ser. No. 63/338,184, filed on May 4, 2022; wherein the entirety of each of said provisional application is incorporated herein by reference.

### BACKGROUND

The present disclosure relates to the production of enzymes useful in the making of one or more bioproducts.

Bioprocessing of feedstocks to produce various bioproducts is an increasingly important source of products including such things as feed, food, fuel, pharmaceuticals, and other chemicals. Many bioprocesses utilize the addition of one or more enzymes which can be an expensive input to the process. Enzymes able to hydrolyze some components can be relatively expensive. There is a continuing need for economical sources of enzymes.

### SUMMARY

The present disclosure includes embodiments of a bioprocessing facility having enzyme production. The bioprocessing facility includes:

a primary fermentation system configured to expose fermentable composition to fermentation conditions and produce beer including one or more bioproducts;

a separation system in fluid communication with the primary fermentation system, wherein the separation system is configured to separate at least a portion of at least one of the one or more bioproducts from beer produced in the primary fermentation system and form whole stillage;

a secondary fermentation system in fluid communication with one or more sources of a stillage composition, wherein the one or more sources of a stillage composition are chosen from whole stillage, stillage derived from whole stillage, and combinations thereof, wherein the secondary fermentation system is configured to form fermentable composition using at least a portion of the one or more sources of a stillage composition and one or more microorganisms, wherein the secondary fermentation system is configured to expose fermentable composition to fermentation conditions so the one or more microorganisms can produce one or more bioproducts and form secondary fermentation broth, wherein the one or more bioproducts include one or more enzymes, and wherein the secondary fermentation system is in fluid communication with one or more systems in the bioprocessing facility so the one or more systems can combine at least a portion of the one or more enzymes with one or more process compositions in the bioprocessing facility to hydrolyze at least one substrate in the one or more process compositions.

The present disclosure also includes embodiments of a method of producing one or more enzymes in a bioprocessing facility. The method includes:

exposing fermentable composition to fermentation conditions in a primary fermentation system to produce beer including one or more bioproducts;

separating at least a portion of at least one of the one or more bioproducts from beer produced in the primary fermentation system to form whole stillage;

exposing fermentable composition to fermentation conditions in a secondary fermentation system so one or more microorganisms can produce one or more bioproducts, wherein the one or more bioproducts include one or more enzymes, wherein fermentable composition in the secondary fermentation system is formed from at least a portion of one or more stillage compositions produced in the bioprocessing facility, wherein the one or more stillage compositions are chosen from whole stillage, a stillage derived from whole stillage, and combinations thereof;

combining at least a portion of the one or more enzymes produced in the secondary fermentation system with one or more process compositions in the bioprocessing facility to hydrolyze at least one substrate in the one or more process compositions.

### BRIEF DESCRIPTION OF THE DRAWINGS

Various examples of the present disclosure will be discussed with reference to the appended drawings. These drawings depict only illustrative examples of the disclosure and are not to be considered limiting of its scope.

FIG. 1A is an illustrative example of a process with integrated enzyme production utilizing one or more stillage compositions as a growth medium for one or more enzyme producing microorganisms;

FIG. 1B is an illustrative example of supplying supplemental feedstock for the enzyme production process shown in FIG. 1A:

FIG. 2 is an another illustrative example of a process with integrated enzyme production utilizing whole stillage as a growth medium for one or more enzyme producing microorganisms;

FIG. 3A is an another illustrative example of a process with integrated enzyme production utilizing thin stillage as a growth medium for one or more enzyme producing microorganisms;

FIG. 3B illustrates an alternative use of a solids stream separated from secondary fermentation broth produced from enzyme production shown in FIG. 3A;

FIG. 3C illustrates supplemental feedstock being used for enzyme production shown in FIG. 3A and also shows whole broth backset being provided to primary fermentation instead of being separated first;

FIG. 3D illustrates an embodiment of the supplemental feedstock shown in FIG. 3C;

FIG. 3E illustrates another embodiment of the supplemental feedstock shown in FIG. 3C;

FIG. 4A shows ethanol production data for Example 1;

FIG. 4B shows oil data for Example 1;

FIG. 5A shows ethanol production data for Example 2;

FIG. 5B shows oil data for Example 2;

FIG. 6A shows ethanol production data for Example 3;

FIG. 6B shows oil data for Example 3; and

FIG. 6C shows residual starch data for Example 3.

### DETAILED DESCRIPTION

The present disclosure relates to producing one or more enzymes on-site at a bioprocessing facility. As used herein, a bioprocessing facility refers to a facility that can produce one or more bioproducts by converting biomass feedstock via one or more physical processes, one or more chemical processes, one or more bioprocesses, and combinations thereof. Non-limiting examples of bioprocessing facilities include dry mills, wet mills, biofuel production facilities, pharmaceutical production facilities, soy processing facilities, breweries, bakeries, and the like.

A bioproduct refers to a product derived from a biological, renewable resource. For example, a bioproduct can be a component of biomass feedstock that is liberated from the biomass feedstock (e.g., corn oil from corn grain) and/or can include a chemical (“biochemical”) that is produced by a biocatalyst (e.g., microorganism and/or enzyme) such as, for example, alcohol produced by yeast fermenting sugar. Non-limiting examples of bioproducts produced in a bioprocessing facility include one or more of fuel, feed, food, pharmaceuticals, beverages and precursor chemicals. In some embodiments, a bioproduct includes, among others, one or more monomeric sugars, one or more enzymes, one or more oils, one or more alcohols (e.g., ethanol, butanol, and the like), fungal biomass, amino acids, and lactic acid.

In some embodiments, one or more bioprocesses are carried out in a bioprocessing facility utilizing living cells (one or more microorganisms) and/or their components (e.g., enzymes produced by a microorganism) to obtain a desired bioproduct. Non-limiting examples of bioprocesses include one or more of hydrolysis (e.g., enzymatic hydrolysis), aerobic fermentation, or anaerobic fermentation. In some embodiments, a bioprocess includes saccharification and fermentation of a plant-based feedstock into a biofuel via enzymatic hydrolysis and yeast-based fermentation of the hydrolysate (e.g., a grain-to-ethanol biofuel facility).

According to one aspect of the present disclosure, enzymes can be produced on-site at a bioprocessing facility that utilizes a bioprocess to produce a biochemical.

In one example, a bioprocessing facility utilizes one or more enzymes to hydrolyze feedstock into monomeric sugars and/or other components that can be converted into the biochemical via a bioprocess yielding a beer (or broth) or to breakdown the feedstock to liberate a valuable bioproduct such as oil. A separation system separates one or more target biochemicals from the beer to yield a biochemical stream and a co-product stream. According to the present disclosure, one or more enzymes can be produced utilizing at least a portion of the co-product stream as growth medium for an enzyme producing microorganism. At least a portion of the one or more enzymes can be supplied to one or more bioprocesses within the bioprocessing facility (e.g., a bioprocess to hydrolyze feedstock). In some embodiments, the enzyme production is integrated into the bioprocess that utilizes the enzyme so that enzymes are produced utilizing a co-product of the bioprocess and an enzyme containing stream is recycled back to the bioprocess.

In some embodiments, at least a portion of a stillage composition is used in a secondary fermentation process in a bioprocessing facility to produce one or more enzymes that are subsequently used within the bioprocessing facility. Advantageously, utilization of a co-product stream (e.g., a stillage composition) for enzyme production may result in one or more benefits such as lower enzyme cost, lower carbon intensity, upgrading of the co-product stream (e.g. reduced fiber, oil, and/or starch, or increased protein for feed or food applications). For example, an enzyme production system according to the present disclosure may use a stillage composition to produce a broth via secondary fermentation, where the broth includes microorganism biomass and enzymes. In some embodiments, as discussed below, a solid-liquid separation of this broth can produce an enzyme-rich liquid portion and a protein-rich solids portion. The liquid portion may be provided to a fermenter, e.g., as enzyme rich backset, to provide enzymes for hydrolyzing components in the biomass feedstock. The protein-rich solids portion may be sold, e.g., wet or dry, as a protein rich feed or food. Further, producing enzymes according to the present disclosure may result in savings by, at least in part, eliminating transportation and/or enzyme product stabilization processes.

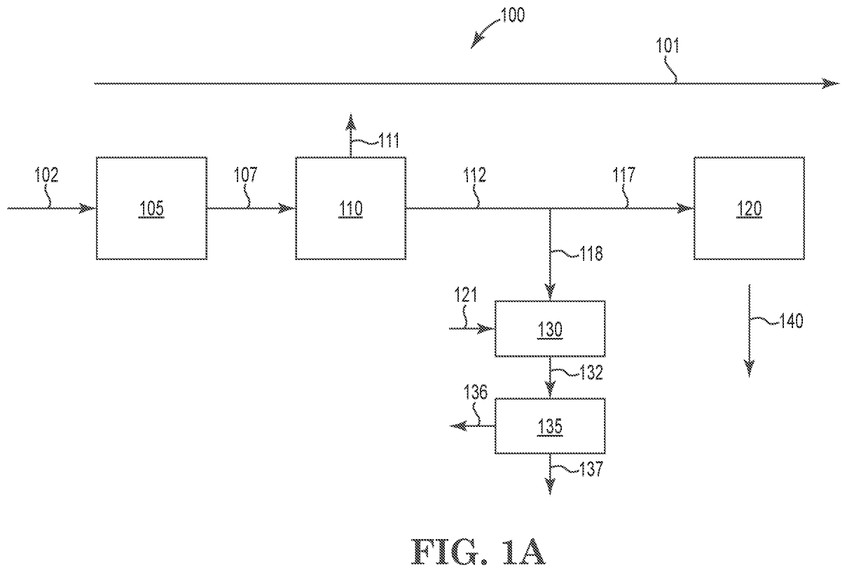

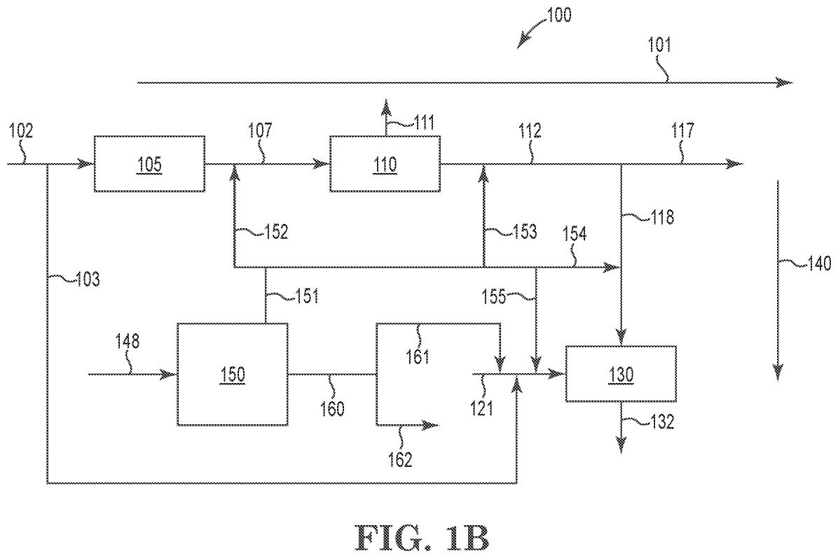

A non-limiting embodiment of the present disclosure is described with reference to FIG. 1A. As shown in FIG. 1A, a bioprocessing facility 100 includes a secondary fermentation system 130 as an ancillary system/process in a secondary process flow 140 that produces one or more enzymes that are used to support the primary process flow 101 of bioprocessing facility 100. Primary process flow 101 includes a primary fermentation system 105 that is in fluid communication with one or more sources 102 of material used to provide a fermentable composition that can ferment in primary fermentation system 105.

Non-limiting examples of a source of material to provide a fermentable composition include one or more of microorganisms, enzymes, carbon sources (e.g., feedstock), aqueous compositions (e.g., fresh water, backset, etc.), nutrient sources (e.g., feedstock), etc. In some embodiments, a feedstock can function as a carbon source and/or a nutrient source, and can be used to form a fermentable composition. A feedstock can include one or more components that are utilized by a microorganism to produce one or more bioproducts via a bioprocess. Non-limiting examples of a feedstocks can be derived from biomass (e.g., plant-based) and may include, e.g., monosaccharides such as glucose and fructose, disaccharides such as sucrose and lactose, and more complex polysaccharides such as starch, cellulose, hemicellulose, and pectin. These biomass-derived feedstocks may come from the seed, sap, stems, and leaves of plants. A wide variety of plant-based feedstocks can be used according to the present disclosure such as sugar beets, sugar cane, grains, legumes, crop residues (e.g., husks, stems, corn stover, sugarcane bagasse, wheat straw), grasses, and woody plants. In some embodiments, feedstock can be derived from corn, sorghum, wheat, rice, barley, soybean, rapeseed, oats, millet, rye, corn stover, straw, bagasse and the like. In some embodiments, a feedstock can include whole ground grain (e.g., corn flour) formed via, e.g., a dry-grind process.

As discussed below, a feedstock can include biomass that has been processed through at least one fermentation such as primary fermentation. A non-limiting example of such feedstock includes one or more stillage compositions.

In some embodiments, a bioprocessing facility can include one or more feedstock systems to process feedstock from one form into another form prior to fermentation. For example, a feedstock system can include one or more size-reduction devices to reduce the size of raw feedstock such as grain and/or further reducing the size of ground grain that has previously been reduced in size. Methods for reducing the size of feedstock, e.g., corn and/or previously ground corn, into fine particles prior to fermentation include dry methods such as passing corn through one or more hammer mills, ball mills and/or roller mills or wet methods such as passing a ground grain slurry through one or more mills such as a disc mill, roller mill, colloid mill, ball mill or other type of milling device.

In some embodiments, a ground feedstock can be mixed with an appropriate amount of water (e.g., in a slurry tank) to form at least a portion of a fermentable composition (sometimes referred to as a mash). In some embodiments, whole ground corn can be mixed with liquid at about 20 to about 50 wt-% or about 25 to about 45 wt-% dry whole ground corn based on the total weight of the slurry. Whole ground corn can include starch, fiber, protein, oil, endogenous enzymes, amino acids, etc.

One or more exogenous microorganisms can be present in the fermentable composition of the primary fermentation system to produce beer that includes at least one or more biochemical bioproducts. Primary fermentation by a microorganism can produce biomass (e.g., single cell protein (SCP)), extracellular metabolites (e.g., alcohol such as ethanol), intracellular metabolites (e.g., enzymes), and combinations thereof. Non-limiting examples of such microorganisms include one or more of ethanologens, butanologens, and the like. Exemplary microorganisms include one or more of yeast, algae, or bacteria. For example, yeast may be used to convert the sugars to an alcohol such as ethanol. Suitable yeast includes any variety of commercially available yeast, such as commercial strains of Saccharomyces cerevisiae.

Optionally, one or more additional, exogenous materials may be utilized in a fermentable composition. Non-limiting examples of such materials include one or more of enzymes, phosphate, citric acid, ionic additives, and the like. In some embodiments, as described below, at least a portion to enzymes used for enzymatic hydrolysis of material for use in a fermentable composition in primary fermentation can be provided at least in part by a secondary fermentation according to the present disclosure.

A primary fermentation system according to the present disclosure can include one or more vessels that are adapted to expose a fermentable composition to conditions suitable for converting sugars such as glucose to one or more bioproducts. As used herein, a “vessel” refers to any vessel that permits a bioproduct to be formed from a microorganism via fermentation. In some embodiments, a vessel can refer to a bioreactor adapted or configured to expose a fermentable composition to fermentation conditions. Non-limiting examples of vessels that can expose a fermentable composition to fermentation conditions include fermenters, beer wells, and the like. Two or more vessels may be arranged in any desired configuration such as parallel or series.

A primary fermentation system is configured to expose fermentable composition to fermentation conditions so that one or more microorganisms can convert one or more components in the fermentable composition such as sugars into a beer that includes one or more target bioproducts. Fermentation conditions include one or more conditions such as pH, time, temperature, aeration, stirring, and the like.

The pH of a fermentable composition can be at a pH that helps a microorganism produce one or more target bioproducts in a desired quantity. In some embodiments, the pH is greater than 3.5, e.g., from 3.5 to 7, from 3.5 to 5.5, or even from 3.5 to 4.5. Techniques for adjusting and maintaining pH include, e.g., adding one or more acidic materials and/or adding one or more basic materials.

With respect to temperature and time, the contents of a fermentable composition can be maintained at temperature for time period helps a microorganism produce one or more target bioproducts in a desired quantity. In some embodiments, the temperature of a fermentable composition can be at a temperature in a range from 20° C. to 45° F., from 25° C. to 40° C., or even from 30° C. to 40° C. In some embodiments, primary fermentation can occur for a time period up to 72 hours, e.g., from 1 hour to 48 hours, from 2 hours to 48 hours, or even from 10 hours to 30 hours.

Primary fermentation can be performed under anaerobic conditions and/or aerobic conditions. For example, primary fermentation can be performed under aerobic conditions for at least a portion of the primary fermentation and performed under anaerobic conditions for another portion of primary fermentation. Alternatively, all of primary fermentation can be performed under anaerobic conditions or under aerobic conditions. Anaerobic or aerobic conditions are selected based on the “target” biochemical or biochemicals chosen to be produced by a microorganism even though there may de minimis amounts of “non-target” biochemicals that are also produced by the microorganism. Anaerobic conditions means that the fermentation process is conducted without any intentional introduction of oxygen-containing gases such as with equipment like blowers, compressors, etc., that could operate to create an aerobic environment suitable for aerobic fermentation. It is noted that while simply stirring a fermentable composition to keep reactor contents homogenous may or may not introduce a de minimis amount of an oxygen-containing gas such as air in some embodiments, stirring alone may not create conditions that would be considered “aerobic conditions” as used herein. However, if desired, the contents of a fermenter could be mixed using appropriate equipment such that sufficient oxygen is introduced throughout the fermentable composition to create an aerobic environment suitable for aerobic fermentation (see below).

Aerobic conditions means that fermentation is performed with intentional introduction of one or more oxygen-containing gasses (“aeration”) to create an aerobic environment suitable for aerobic fermentation so that oxygen can be consumed by one or more microorganisms and selectively favor the production of enzymes via an aerobic metabolic pathway as compared to an anaerobic pathway which favors production of biochemicals (e.g., alcohol, organic acids, and the like). A fermentation system may incorporate aeration by including one or more blowers, spargers, gas compressors, mixing devices, and the like, that are in fluid communication with one or more fermentation vessels and that can introduce an oxygen-containing gas (e.g., air) into a fermentable composition during at least a portion of fermentation. For example, an oxygen-containing gas can be sparged into a fermentable composition so that the gas bubbles up and through the fermentable composition and oxygen transfers into the fermentable composition. As another example, an oxygen-containing gas can be introduced into the headspace of a fermenter so that the gas diffuses into the fermentable composition.

Optionally, in addition to aeration, a fermentable composition can be agitated or mixed to facilitate transferring oxygen into and throughout the fermentable composition so as to achieve an aerobic environment. For example, a continuous stirred tank reactor (CSTR) can be used to agitate or mix the fermentable composition. The speed of the stirring mechanism (rpms) can be adjusted based on a variety of factors such as tank size, slurry viscosity, and the like. As mentioned above, in addition to mixing the contents of a composition, mixing can be selected, if desired, to intentionally incorporate oxygen to a fermentable composition to facilitate aerobic fermentation.

A primary fermentation system can be operated according to batch fermentation, fed-batch fermentation, or continuous fermentation (continuous feed and discharge from a vessel such as a fermenter).

Also, a fermentation system according to the present disclosure can conduct fermentation sequentially or simultaneously with respect to a polysaccharide hydrolysis/saccharification process (e.g., jet-cooking and/or enzymatic hydrolysis). Saccharification and fermentation can occur simultaneously according to what is known as “simultaneous saccharification and fermentation” (“SSF”). Sequential hydrolysis and fermentation can also be referred to as separate hydrolysis and fermentation (SHF).

An example of an SSF is described below in the context of a starch-containing grain such as corn. A slurry (“grain mash composition”) can be combined with a microorganism to form a fermentable composition so that at least a portion of starch in the fermentable composition is hydrolyzed by one or more enzymes to produce monosaccharides. As the monosaccharides are produced they can be metabolized by a microorganism into a target biochemical product. For example, sugar (glucose, xylose, mannose, arabinose, etc.) that is generated from saccharification can be fermented into one or more biochemicals (e.g., butanol, ethanol, and the like).

Alternatively, an SHF process may include a dedicated saccharification process that is separate from a fermentation process (either in the same or separate vessel). For example, after forming an aqueous slurry that includes the biomass feedstock (e.g. corn material from a milling system) the aqueous slurry can be subjected to saccharification in one or more slurry tanks to break down (hydrolyze) at least a portion of the polysaccharides, e.g. starch, cellulose, hemicellulose, etc., into oligosaccharides and/or monosaccharides (e.g. glucose, xylose, mannose, arabinose, etc.) that can be used by microorganisms (e.g., yeast) in a subsequent fermentation process.

Saccharification can be performed by a variety of mechanisms. For example, heat and/or one or more enzymes can be used to form one or more monosaccharides by saccharifying one or more oligosaccharides and/or one or more polysaccharides that are present in a polysaccharide such as starch. In some embodiments, a relatively low temperature saccharification process (whether used in SSF or SHF) involves enzymatically hydrolyzing at least a portion of starch in an aqueous slurry at a temperature below starch gelatinization temperatures, so that saccharification occurs directly from the raw native insoluble starch to soluble glucose while bypassing conventional starch gelatinization conditions, which are typically in a range of 57° C. to 93° C. depending on the starch source and polysaccharide type. In some embodiments, saccharification includes using one or more enzymes (e.g., alpha-amylases and/or gluco-amylases) to enzymatically hydrolyze at least a portion of the starch in the aqueous slurry at a temperature of 40° C. or less to produce a slurry that includes glucose. In some embodiments, enzymatic hydrolysis occurs at a temperature in the range of from 25° C. to 35° C. to produce a slurry that includes glucose. Non-limiting examples of converting raw starch to glucose are described in U.S. Pat. No. 7,842,484 (Lewis), U.S. Pat. No. 7,919,289 (Lewis), U.S. Pat. No. 7,919,291 (Lewis et al.), U.S. Pat. No. 8,409,639 (Lewis et al.), U.S. Pat. No. 8,409,640 (Lewis et al.), U.S. Pat. No. 8,497,082 (Lewis), U.S. Pat. No. 8,597,919 (Lewis), U.S. Pat. No. 8,748,141 (Lewis et al.), 2014-0283226 (Lewis et al.), and 2018-0235167 (Lewis et al.), wherein the entirety of each patent document is incorporated herein by reference.

After fermentation in a primary fermentation system, one or more bioproducts can be separated from beer to form at least one target bioproduct stream (e.g., ethanol) and one or more co-product streams (e.g., whole stillage). A separation system according to the present disclosure can be configured to separate at least a portion of at least one of the one or more bioproducts produced in the primary fermentation system. Referring to FIG. 1A, a separation system 110 is in fluid communication with the primary fermentation system 105 to receive a beer stream 107, which is separated into at least one target biochemical stream 111 (e.g., ethanol) and at least one co-product stream 112. For illustration purposes, co-product stream 112 can generically encompass any stillage composition downstream from separation system 110. Stillage compositions are discussed below.

A separation system 110 according to the present disclosure can separate a bioproduct from a beer using one or more of distillation, evaporation, separation based on particle size (e.g., filtration), or separation based on density (e.g., centrifugation). In some embodiments, a separation system 110 can include one or more centrifuges (e.g., two-phase vertical disk stack centrifuge, three-phase vertical disk stack centrifuge, filtration centrifuge), one or more decanters (e.g., filtration decanters), one or more filters (e.g., fiber filter, rotary vacuum drum filter, filter device having one or more membrane filters), one or more screens (e.g., a “DSM” screen, which refers to a Dutch State Mines screen or sieve bend screen, and is a curved concave wedge bar type of stationary screen; a pressure screen; paddle screen; rotary drum screen; centrifugal screener; linear motion screen; vacu-deck screen; etc.), one or more brush strainers, one or more vibratory separators, one or more hydrocyclones, one or more presses, combinations of these and the like. Multiple separation systems can be used together and arranged in a parallel and/or series configuration. Depending on the separation system selected, one or more process input streams can be separated into two or more output streams to produce an output stream that has a higher amount of solids as compared to other output streams. If desired, a separation system can include one or more evaporators and/or one or more dryers to further concentrate an output stream from any of the devices just mentioned.

According to the present disclosure, at least a portion of the one or more co-product streams derived from beer can be used as feedstock for secondary fermentation to produce one or more enzymes. In some embodiments, by using co-product as a feedstock the co-product can function as a carbon source and/or a nutrient source for one or more microorganisms in a fermentable composition of the secondary fermentation system. In some embodiments, the co-product can include one or more stillage compositions and a secondary fermentation system according to the present disclosure is configured to form fermentable composition using at least a portion of the one or more stillage compositions.

As used herein, a “stillage composition” can include whole stillage, at least one stillage composition derived from whole stillage, and combinations thereof. Non-limiting examples of a stillage composition derived from whole stillage include thin stillage, concentrated thin stillage (syrup), defatted syrup, defatted emulsion, clarified thin stillage, distiller's oil, distiller's grain, distiller's yeast, and the like. Non-limiting examples of defatted stillage compositions include one or more defatted streams derived from thin stillage such as defatted syrup, defatted emulsion, and the like. In some embodiments, a stillage composition can be exposed to an intermediate fermentation (not shown) after primary fermentation but prior to a secondary fermentation that produces enzymes as described herein.

Referring to FIG. 1A, a portion 118 of a stillage composition is separated as a “slip stream” from the primary process flow 101 and is in fluid communication with secondary fermentation system 130 so that the portion 118 of the stillage composition 112 can be used in a secondary process flow 140 as at least part of the feedstock for fermentation in the secondary fermentation 130. Because portion 118 can be obtained from whole stillage and/or one or more stillage compositions derived from whole stillage, portion 118 may be obtained from one or more locations along the primary process flow 101.

The remaining portion 117, if any, of the stillage composition 112 that portion 118 is separated from may be processed in one or more sub-systems 120 of the primary process flow 101 of the bioprocessing facility 100 to separate co-products such as oil, protein, and the like. Non-limiting examples of methods and systems for processing stillage streams are described in U.S. Pat. No. 8,702,819 (Bootsma); U.S. Pat. No. 9,061,987 (Bootsma); U.S. Pat. No. 9,290,728 (Bootsma); U.S. Pat. No. 10,059,966 (Bootsma); U.S. Pat. No. 11,248,197 (Bootsma); and U.S. Pub. No. 2020/0140899 (Bootsma); wherein the entirety of each of said patent documents is incorporated herein by reference.

In some embodiments, secondary fermentation system 130 is in fluid communication with one or more sources 121 of material, in addition to one or more stillage compositions 118, that can be used to provide a fermentable composition that can ferment in secondary fermentation system 130. Non-limiting examples of such material include one or more of microorganisms, enzymes, carbon sources (e.g., feedstock), aqueous compositions (e.g., fresh water, backset, etc.), nutrient sources (e.g., feedstock), etc. Such material can be the same or different as used in primary fermentation system 105. One or more sources 121 may include supplemental feedstock provided by a feedstock system 150, which is discussed below with respect to FIG. 1B.

Secondary fermentation by a microorganism can produce biomass (e.g., single cell protein (SCP), extracellular metabolites (e.g., alcohol such as ethanol), intracellular metabolites (e.g., enzymes), and combinations thereof. One difference between primary fermentation system 105 and secondary fermentation system 130 may be the one or more microorganisms used in secondary fermentation system 130. A fermentable composition used in secondary fermentation system 130 can include one or more microorganisms that produce one or more target enzymes. Secondary fermentation according to the present disclosure can produce a wide variety of enzymes as a bioproduct. Non-limiting examples of enzymes useful in one or more bioprocesses include one or more of hydrolases or carbohydrases. In some embodiments, an enzyme produced during secondary fermentation and that is useful in one or more bioprocesses includes one or more of proteases, lipases, esterases, phytases, amylases, cellulases, hemicellulases, pectinases, etc. As mentioned, secondary fermentation can produce one or more enzymes. Multiple enzymes can be from the same type/class of enzyme with similar activity (e.g., multiple cellulases) and/or of different types/classes (e.g., at least one cellulase and at least one hemicellulose). In more detail, secondary fermentation can produce multiple different enzymes with similar activity, e.g., multiple different cellulases, multiple different proteases, multiple different lipases, multiple different esterases, multiple different phytases, multiple different alpha-amylases, multiple different gluco-amylases, multiple different cellulases, multiple different hemicellulases, multiple different pectinases, multiple different xylanases, etc., and combinations thereof. Without being bound by theory, it is believed that an enzyme broth produced in secondary fermentation and that has multiple different enzymes of similar activity can improve performance of the enzyme broth in a subsequent process (e.g., primary fermentation).

In some embodiments, certain enzymes can be relatively expensive and tend to be cost prohibitive if they are produced at an enzyme manufacturing facility external to bioprocessing facility 100 and added, e.g., to a biofuel fermentation system such as 105 at bioprocessing facility 100. Such enzymes may be beneficial to a fermentation process and/or to recovery of one or more co-products, but the revenue generated may not cover the costs of using the enzymes. Furthermore, traditional enzyme production at an enzyme manufacturing facility may contribute to the carbon intensity of bioprocesses using the enzymes. In some embodiments, such enzymes include “accessory” (non-amylase) enzymes such as cellulases, xylanases, and other non-amylases. For example, using one or more accessory enzymes in primary fermentation system 105 can improve separation of one or more co-products (e.g., oil, protein, and the like) from the remaining portion 117 of the stillage composition in one or more sub-systems 120 of the primary process flow 101 of the bioprocessing facility 100. In another example, using one or more accessory enzymes in primary fermentation system 105 can improve feedstock utilization by hydrolyzing recalcitrant portions of the feedstock (e.g., fiber).

Non-limiting examples of enzyme producing microorganisms include one or more of bacteria, algae, and fungi (e.g., yeast, filamentous fungi, etc.). In some embodiments, enzyme producing microorganisms may include Trichoderma reesei and/or Bacillus subtilis. Non-limiting examples of microorganisms that may be engineered to produce enzymes include one or more of Saccharomyces cerevisiae, Cyberlindnera jadinii, Lactobacillus strains, Escherichia coli, Zymomonas mobilis, Candida utilis, Aspergillus niger, Kluyveromyces marxianus, Yarrowia hpolytica, Candida tropicalis, Candida albican, and Scheffersomyces stipites. Microorganisms may or may not excrete the enzyme or enzymes that are produced. As discussed below, in some embodiments, microorganisms may be exposed to a lysis process after secondary fermentation to release the enzymes produced.

As mentioned, secondary fermentation can be performed using one or more microorganisms. For example, multiple enzymes may be produced by using multiple microorganisms. As another example, multiple pathways in a single microorganism may be employed to produce multiple different enzymes with similar activity, e.g., multiple different cellulases, multiple different proteases, multiple different lipases, multiple different esterases, multiple different phytases, multiple different alpha-amylases, multiple different gluco-amylases, multiple different cellulases, multiple different hemicellulases, multiple different pectinases, multiple different xylanases, etc. In some embodiments, secondary fermentation may “co-culture” more than one microorganism in a fermentation vessel to produce multiple enzymes. In some embodiments, secondary fermentation may include multiple fermentation vessels with each vessel operated to produce different enzymes, e.g. by growing different microorganisms. In some embodiments, a single microorganism may produce more than one enzyme in during secondary fermentation.

In some embodiments, secondary fermentation may provide a co-culture of an enzyme-producing microorganism that produces one or more organic acids along with enzymes and another microorganism that metabolizes the organic acids to prevent the organic acids from accumulating in the enzyme broth. Avoiding the accumulation of one or more organic acids in a secondary fermentation broth can help avoid inhibiting a microorganism present in secondary fermentation and/or a microorganism that is present in a composition that the secondary fermentation broth is incorporated into (e.g., primary fermentation).

A secondary fermentation system according to the present disclosure can include one or more vessels that are adapted to expose a fermentable composition to conditions suitable for converting sugars such as glucose to one or more bioproducts. A “vessel” with respect to a secondary fermentation system is as similarly explained above with respect to primary fermentation system. In some embodiments, referring to FIG. 1A, since secondary fermentation system 130 is an ancillary system/process in a secondary process flow 140, it may be operated on a relatively smaller scale and/or on an intermittent basis as compared to primary fermentation system 105, if desired.

A secondary fermentation system according to the present disclosure is configured to expose fermentable composition to fermentation conditions so that microorganisms present in the fermentable composition produce one or more enzymes. Fermentation conditions include one or more conditions such as pH, time, temperature, aeration, stirring, and the like. Fermentation conditions for the secondary fermentation system 130 may be the same or different from fermentation conditions of the primary fermentation system 105. The fermentation conditions for the secondary fermentation system depend on a variety of factors, including the microorganism or microorganisms selected for fermentation.

The pH of a fermentable composition in secondary fermentation can be at a pH that helps a microorganism produce one or more enzymes in a desired quantity. In some embodiments, the pH is greater than 4, or even greater than 5.5, e.g., from 5.5 to 7, or even from 5.5 to 6.5. In some embodiments, a stillage composition such as whole stillage or thin stillage may be at a pH that is too low for desirable performance by a microorganism so the pH may have to be adjusted upward. For example, in some embodiments, the pH may have to be adjusted from about 4 to at least 5.5. Techniques for adjusting and maintaining pH include, e.g., adding one or more acidic materials and/or adding one or more basic materials.

With respect to temperature and time, the contents of a fermentable composition in secondary fermentation can be maintained at temperature for time period helps a microorganism produce one or more enzymes in a desired quantity. In some embodiments, the temperature of a fermentable composition in secondary fermentation can be at a temperature in a range from 20° C. to 45° F., from 25° C. to 40° C., or even from 30° C. to 40° C. In some embodiments, secondary fermentation can occur for a time period up to 72 hours, e.g., from 1 hour to 48 hours, from 2 hours to 48 hours, or even from 10 hours to 30 hours.

Like primary fermentation discussed above, secondary fermentation can be performed under anaerobic conditions and/or aerobic conditions. For example, secondary fermentation can be performed under aerobic conditions for at least portion of secondary fermentation and performed under anaerobic conditions for another portion of secondary fermentation. Alternatively, all of secondary fermentation can be performed under anaerobic conditions or under aerobic conditions. Optionally, in addition to aeration, a fermentable composition can be agitated or mixed to facilitate transferring oxygen into and throughout the fermentable composition so as to achieve an aerobic environment. For example, a continuous stirred tank reactor (CSTR) can be used to agitate or mix the fermentable composition. The speed of the stirring mechanism (rpms) can be adjusted based on a variety of factors such as tank size, slurry viscosity, and the like. As mentioned above, in addition to mixing the contents of a composition, mixing can be selected, if desired, to intentionally incorporate oxygen to a fermentable composition to facilitate aerobic fermentation.

A secondary fermentation system can be operated according to batch fermentation, fed-batch fermentation, or continuous fermentation (continuous feed and discharge from a vessel such as a fermenter).

Also, a secondary fermentation system according to the present disclosure can conduct fermentation sequentially (SHF) or simultaneously (SSF) with respect to a polysaccharide hydrolysis/saccharification process (e.g., jet-cooking and/or enzymatic hydrolysis). SHF and SSF are discussed above with respect to primary fermentation and are not repeated here.

According to the present disclosure, at least a portion of the enzyme or enzymes produced in a secondary fermentation system can be subsequently used in one or more bioprocesses within the bioprocessing facility that produces the enzymes. As such, a secondary fermentation system according to the present disclosure is in fluid communication with one or more systems in the bioprocessing facility so that the one or more systems can receive and combine at least a portion of the one or more enzymes with one or more process compositions in the bioprocessing facility to hydrolyze one or more substrates in the one or more process compositions. A process composition in a bioprocessing facility can include any process composition that can utilize the produced enzyme or enzymes. As mentioned above, enzyme produced during secondary fermentation can include one or more proteases, lipases, esterases, phytases, amylases, cellulases, hemicellulases, pectinases, etc. Accordingly, a process composition that includes a substrate that can be hydrolyzed by enzyme produced in secondary fermentation may benefit from receiving enzyme produced in the secondary fermentation system 130. Non-limiting examples of such substrates include starch, cellulose, hemicellulose, pectin, amylase, amylose, fat, protein, and the like. Non-limiting examples of process compositions that may include such substrates include a composition upstream of a fermenter in a primary fermentation system (e.g., a slurry for use in fermenter), a fermentable composition within a fermenter of the primary fermentation system, a beer well, a stillage composition, and the like.

In some embodiments, a process composition in a bioprocessing facility can be directly combined with the enzyme that is produced in the secondary fermentation system. For example, enzyme produced in secondary fermentation system 130 may be added directly into a slurry upstream from primary fermentation 105 and/or directly into a fermenter in primary fermentation system 105.

In some embodiments, a process composition can be downstream from and derived from a process composition where the enzyme is introduced. In this situation the enzyme produced is said to be in “indirect” fluid communication with the process composition. For example, enzyme produced in secondary fermentation system 130 may be added directly into a slurry upstream from primary fermentation 105 and, therefore, the broth produced in secondary fermentation system 130 is considered in indirect fluid communication with a fermentable composition present in a fermenter in primary fermentation system 105.

Referring to FIG. 1A, enzyme produced in the secondary fermentation system 130 can be in direct and/or indirect fluid communication with one or more fermenters in the primary fermentation system 105, thereby allowing at least a portion of the contents, including enzymes, from a fermenter in the secondary fermentation system 130 to be conveyed to one or more fermenters in the primary fermentation system 105 as backset. For example, enzyme produced in secondary fermentation system 130 may be introduced into a slurry tank and the slurry may be transferred to a fermenter in primary fermentation system 105 where the majority of the enzymatic activity occurs. In some embodiments, enzyme production in secondary fermentation system 130 can lower the solids in backset (e.g., 132 or 136, which are discussed below) recycled to the primary fermentation system 105. If desired, because of lowered solids, the amount of feedstock added to primary fermentation system 105 can be increased, which generally translates to increased production of one or more bioproducts in the primary process flow 101.

In some embodiments, referring to FIG. 1A, enzyme produced in the secondary fermentation system 130 is in direct and/or indirect fluid communication with one or more process compositions downstream from primary fermentation system 105. For example, at least a portion of enzyme produced in the secondary fermentation system 130 can be introduced into a process composition in the primary process flow 101 for enzymatic hydrolysis and/or one or more secondary fermentations that may be performed in the primary process flow 101 downstream from the primary fermentation system 105. As another example, at least a portion of enzyme produced in the secondary fermentation system 130 could be recycled in a manner that permits use of the enzyme in the secondary fermentation system 130.

In some embodiments, enzyme produced in secondary fermentation system 130 can be present within whole broth and the whole broth can be used for at least a portion of backset in a later fermentation. Referring to FIG. 1A enzyme produced in secondary fermentation system 130 can be present within whole broth 132. In some embodiments, a “pre-blend” is used to form a corn mash/slurry for primary fermentation system 105. A pre-blend is referred to as the source or sources of water used to make the corn mash. In some embodiments, at least a portion of whole broth 132 can be used as backset for primary fermentation system 105 and can form 100 percent of the pre-blend. In some embodiments, at least a portion of whole broth 132 can be combined with one or more other sources of water to form a pre-blend. For example, at least a portion of whole broth 132 can form at least 5%, at least 10%, at least 20%, at least 30%, at least 40%, at least 50%, at least 60%, at least 70%, at least 80%, or even at least 90% of the pre-blend based on volume. In some embodiments, at least a portion of whole broth 132 can form between 40 and 80%, or even between 45 and 75% of the pre-blend based on volume.

In some embodiments, enzyme produced in secondary fermentation system can be separated from the whole broth 132 prior to using the enzyme. Referring to FIG. 1A, an optional separation system 135 is in fluid communication with whole broth 132 produced in the secondary fermentation system 130 and is configured to separate at least a portion of the one or more enzymes from whole broth 132. The separation system 135 can separate whole broth 132 into at least one relatively low solids liquid phase enzyme composition 136 and a relatively high solids composition 137. In some embodiments, the relatively high solids composition 137 can used as or to make a feed product as a separate co-product, e.g., a protein feed comprising cellular biomass.

A separation system 135 can use one or more separation techniques to separate a relatively enzyme-rich composition from whole broth produced in secondary fermentation system 130. For example, a separation system 135 can include one or more centrifuges (e.g., two-phase vertical disk stack centrifuge, three-phase vertical disk stack centrifuge, filtration centrifuge), one or more decanters (e.g., filtration decanters), one or more filters (e.g., fiber filter, rotary vacuum drum filter, filter device having one or more membrane filters), one or more screens (e.g., a “DSM” screen, which refers to a Dutch State Mines screen or sieve bend screen, and is a curved concave wedge bar type of stationary screen; a pressure screen; paddle screen; rotary drum screen; centrifugal screener; linear motion screen; vacu-deck screen; etc.), one or more brush strainers, one or more vibratory separators, one or more hydrocyclones, one or more presses, combinations of these and the like, to separate an aqueous enzyme composition (e.g., a clarified broth) from solids in a whole broth to concentrate the enzymes. Multiple separation systems can be used together and arranged in a parallel and/or series configuration. Depending on the separation system selected, one or more input streams can be separated into two or more output streams to provide an output stream having a higher amount of solids as compared to other output streams.

Optionally, whole broth 132 or a composition separated from whole broth 132 can be exposed to a lysis system/process to form a lysate. A lysate can be formed by lysing the microorganism that expresses the enzyme or enzymes that are produced. Lysis of a microorganism refers to cellular disruption that results in the outer boundary or cell membrane being broken down or destroyed in order to release inter-cellular materials. Lysing a microorganism can release enzyme, thereby making the enzyme relatively more available for hydrolysis of a substrate in a process composition in a bioprocessing facility. In addition, lysing a microorganism essentially kills the microorganism, which at least in some embodiments can advantageously help avoid competition (e.g., competition for consuming a carbon source such as sugar) between the microorganism from secondary fermentation and any other microorganism (e.g., ethanologen in primary fermentation). In some embodiments, whole broth from secondary fermentation can be processed to lyse the enzyme producing microorganisms and be provided as a lysed whole broth or separated in separation system 135 to provide a clarified, lysed whole broth.

In some embodiments, a microorganism may release enzyme such that a lysis system may not be used if desired.

A variety of techniques can be used in a lysis system for lysing a microorganism. Methods of lysis are available on both a macro and micro scale. Lysis can be performed using one or more mechanical methods, one or more non-mechanical methods, and combinations thereof. In some embodiments, a recycle loop can be used to recycle the output of a lysis operation and repeatedly expose it to the lysis operation until a fully lysed product is achieved.

In some embodiments, the whole broth of a secondary fermentation system can be cooled and/or concentrated prior to lysing (e.g., via separation as discussed above). Further, the temperature can be controlled throughout lysis so that the temperature does not go above a threshold temperature (i.e. the temperature stays below the melting point of the enzyme) that inactivates the enzymes to an undue degree, thereby resulting in an ineffective lysate. Similarly, the temperature of the enzyme can be controlled to be low enough during any other process or storage that occurs between secondary fermentation and subsequent use to avoid inactivating the enzymes to an undue degree. For example, a temperature control system could be included that maintains a lysate in a frozen condition until use. In some embodiments, the lysis system is configured to control the temperature of the enzyme present in the lysis system to be 140° F. or less, 130° F. or less, 120° F. or less, 110° F. or less, or even 100° F. or less. A lysis system according to the present disclosure can be configured in a variety of ways to control the temperature throughout lysis as described. In some embodiments, a lysis system can include one or more fluid-jacketed vessels that are connected to one or more of a cooling tower water, heat exchanger, or one or more other temperature-controlled water sources at a bioprocessing facility.

Non-limiting examples of mechanical lysing include using homogenizers, bead mills and the like. A homogenizer such as a high-pressure homogenizer (HPH) is an example of equipment for microbial disruption. In an HPH, cells in media are forced through an orifice valve using high pressure causing disruption of the cell membrane due to high shear force at the orifice when the cell is subjected to compression while entering the orifice and expansion upon discharge. Lysis with an HPH can involve passing microorganisms through an HPH one or more times depending on the pressure selected and/or microorganism involved. For example, in some embodiments, yeast can be passed through an HPH at a pressure from 1000 to 1200 bar to cause a sufficient degree of lysis. In some embodiments, bacteria can be lysed at lower pressures. As another example, in some embodiments, microorganisms can be passed through an HPH at a pressure less than 1000 bar (e.g., from 650 to less than 1000 bar) multiple times to cause a sufficient degree of lysis. A bead mill involves disrupting cells by agitating tiny beads made of glass, steel, or ceramic along with a cell suspension at high speeds. The beads collide with the cells and break open the cell membrane and releasing the intracellular components by shear force. Non-mechanical lysing methods include one or more physical methods of lysing, one or more chemical methods of lysing, and one or more biological methods of lysing.

Physical lysing refers to non-contact methods that utilize one or more external forces such as heat, pressure, and sound energy, to rupture the cell membrane. Non-limiting examples of physical lysing include thermal lysis, cavitation, osmotic shock, and the like. Thermal lysis involves repeated freezing and thawing cycles, which cause formation of ice on the cell membrane and helps break down the cell membrane. Cavitation involves the formation and subsequent rupture of cavities or bubbles. Such cavities can be formed by reducing the local pressure which can be done by increasing the velocity, ultrasonic vibration, and the like. Reduction of pressure causes the collapse of the cavity and a large amount of mechanical energy to be released in the form of a shockwave that propagates through the medium. The high energy of the shock wave can disintegrate the cell membrane. Ultrasonic sound and hydrodynamic methods can be used for generating cavitation used to disrupt cells.

Chemical lysing refers to using lysis buffers to disrupt the cell membrane. Non-limiting examples of chemical lysing include alkaline lysis, and detergent lysis. Alkaline lysis can break the cell membrane by changing the pH. Detergents can be added in detergent lysis to solubilize the membrane proteins and to rupture the cell membrane.

Non-limiting examples of biological lysing include using one or more enzymes such as lysozyme, lysostaphin, zymolase, cellulase, protease or glycanase.

A lysis system can be coupled directly or indirectly to one or more fermenters in the secondary fermentation system and configured to form a lysate with at least a portion of contents from the one or more fermenters. In some embodiments, two or more fermenters in a secondary fermentation system can be multiplexed to a single lysis system such that they are temporally staged and cycled to the lysis system in a manner to produce a lysate. In some embodiments, multiplexing fermenters with a single lysis system permits a lysate to be produced in a just-in-time manner for sequential fermentations among multiple fermenters.

A bioprocessing facility according to the present disclosure that includes a lysis system can tailor the lysis system to use one or more lysis techniques in a manner that facilitates just-in-time delivery of the lysate and/or permits a relatively simple method of providing the lysate. For example, in some embodiments, a lysis system could include one or more lysis techniques that are relatively gentle and/or avoid additional processing of the lysate prior to introducing the lysate into a process composition. In some embodiments, lysing includes mechanically and/or physically lysing a microorganism to form a lysate of the microorganism.

In some embodiments, lysing systems and methods according to the present disclosure do not include chemically lysing and/or biological lysing. For example, not using chemical lysing can avoid having to subsequently process the lysate to alter one or more properties prior to introducing the lysate into a process composition. For example, in some enzyme facilities, more aggressive lysing techniques use chemical lysing agents. The lysate is then typically processed to remove components that would be detrimental to, e.g., fermentation.

Because a secondary fermentation system according to the present disclosure can be co-located at a bioprocessing facility, transportation to the bioprocessing facility can be eliminated so that little or no protease inhibition and/or stabilization is necessary, especially if producing enzymes is performed according to a just-in-time protocol.

If desired, one or more evaporators and/or one or more dryers can be used to further concentrate an output stream such as streams 132, 136, and/or 137.

Optionally, the secondary fermentation system is in fluid communication with one or more sources of feedstock in addition to using at least a portion of one or more stillage compositions 118, as described above. Optional sources of feedstock may be the same or different from the feedstocks described above with respect to primary fermentation system 105. In some embodiments, sources of carbon and/or nutrients in addition to stillage composition may be desired, especially as the efficiency of primary fermentation system 105 increases. For example, primary fermentation system 105 may consume starch such that residual starch in stillage 118 is not sufficient to produce the desired amount or concentration of enzymes in secondary fermentation 130 given the metabolic capability of a microorganism. Thus, referring to FIG. 1B, supplemental feedstock 121 may be used and may be provided from one or more sources, alone or in combination.

For example, as discussed above, one or more sources 102 of material can include feedstock prepared by a feedstock system. In some embodiments, feedstock such as corn grain can be ground more coarsely and fed to primary fermentation system 105. The larger particles will be less efficiently utilized in the primary fermentation so that more residual starch passes through the primary fermentation system 105 and into secondary fermentation 130 via one or more stillage compositions 118. In some embodiments, the amount of ground feedstock of a given grind size such as corn flour can be increased and fed to primary fermentation system 105 so that more residual starch passes through the primary fermentation system 105 and into secondary fermentation 130 via one or more stillage compositions 118.

In some embodiments, an amount of ground feedstock such as corn flour can be fed directly into secondary fermentation 130 where carbohydrate may be hydrolyzed via SSF or SHF to form simple carbohydrates for the enzyme producing microorganism or microorganisms. The corn flour fed into secondary fermentation may be a “slip stream” from the same feedstock system that is used for primary fermentations system 105 and/or from a different source. For example, FIG. 1B shows that a portion 103 of feedstock (e.g., corn flour) 102 used for primary fermentations system 105 may be fed directly to secondary fermentation system 130.

In some embodiments, a bioprocessing facility may include a feedstock system that is configured to form a sugar stream and/or a starch stream that is fed to the secondary fermentation system to form fermentable composition. Referring to FIG. 1B, bioprocessing facility 100 can optionally include a feedstock system 150 that forms a sugar stream or a starch stream 160. Feedstock system 150 can received a raw feedstock 148 such as corn grain and process it to form a sugar or starch stream 160, and a “rejects” stream 151. Raw feedstock 148 may be the same or different from the feedstock used in a feedstock system described above for primary fermentation system 105. As shown in FIG. 1B, at least a portion 161 of sugar or starch stream 160 is provided to secondary fermentation system 130 as supplemental feedstock 121. If portion 161 is a starch stream, the starch can be hydrolyzed as described herein into sugars, which can be used by one or more microorganisms to make enzyme. If portion 161 is a sugar stream, the sugar can be used by one or more microorganisms to make enzyme. In some embodiments, if desired, at least a portion 162 of sugar or starch stream 160 can be used for one or more other purposes either internal or external to bioprocessing facility 100.

In some embodiments, if raw feedstock 148 includes corn grain, the rejects stream 151 may include residual corn grain components such as one or more of corn fiber, corn protein, corn germ, hard endosperm, corn oil, residual corn starch, etc. Optionally, if desired, at least a portion of the rejects stream 151 can be introduced into one or more locations with the primary process flow 101 of bioprocessing facility 100. For example, as shown in FIG. 1B, a portion 152 may be introduced into beer stream 107. As another example, a portion of the rejects stream 151 may be introduced into one or more stillage streams in primary process flow 101. For illustration purposes, as shown in FIG. 1B, a portion 153 may be introduced into stillage stream 112 stream. As yet another example, a portion of the rejects stream 151 may be introduced into the secondary process flow 140. For illustration purposes, as shown in FIG. 1B, a portion 154 may be introduced into stillage stream 118 and/or a portion 155 may be used as supplemental feedstock 121. A portion of the rejects 151 could also be introduced into whole broth stream 132 from secondary fermentation (not shown). Advantageously, by introducing rejects back into the primary process flow 101 and/or the secondary process flow 140, the rejects can be broken down by exposure to process conditions and one or more components of the rejects stream 151 may be separated from primary process flow 101 as a co-product (e.g., oil, etc.). A non-limiting example of converting raw feedstock into a starch stream is described in U.S. Pat. No. 10,793,879 (Redford et al.), wherein the entirety of said patent is incorporated herein by reference. A non-limiting example of converting raw feedstock into a sugar stream is described in U.S. Pat. No. 10,233,466 (Redford), wherein the entirety of said patent is incorporated herein by reference.

In some embodiments, supplemental feedstock 121 may also include one or more sources prepared at facilities external to bioprocessing facility 100 such as sources of sugar and/or starch (e.g., from a wet mill facility). In still other embodiments, supplemental feedstock 121 may include a carbohydrate source such as, e.g., meal, hulls, bran, steep water, molasses, and the like from a facility that processes food, feed, or grains such as beans, peas, rice, wheat, and the like.

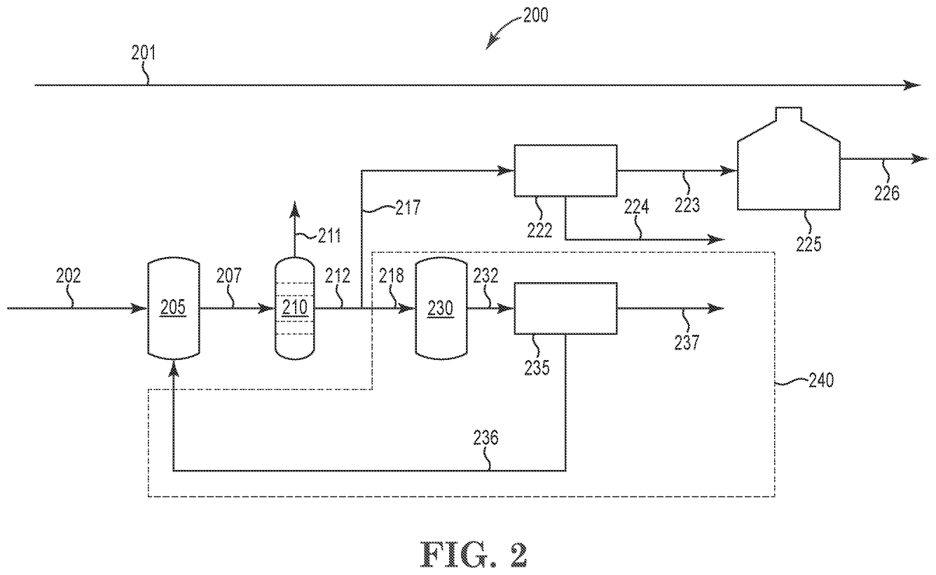

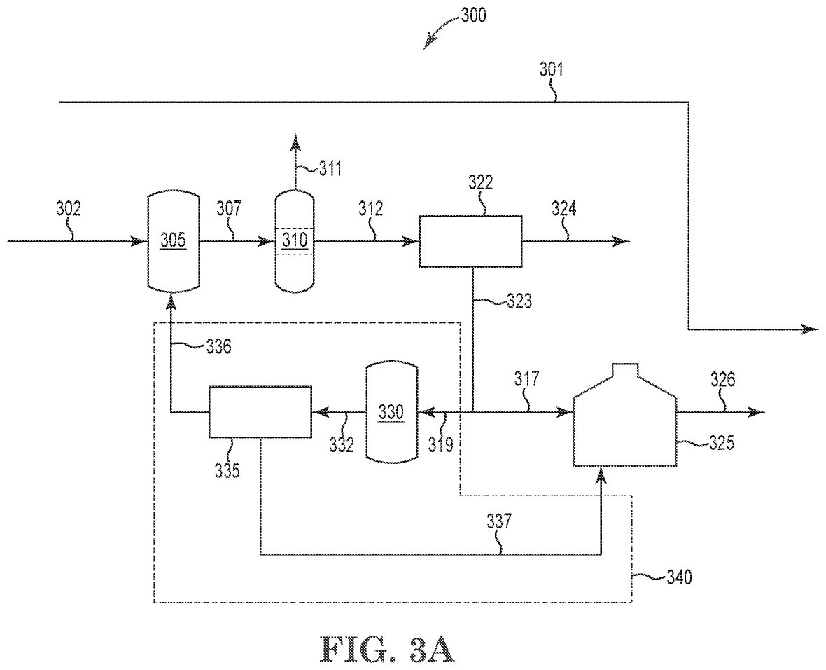

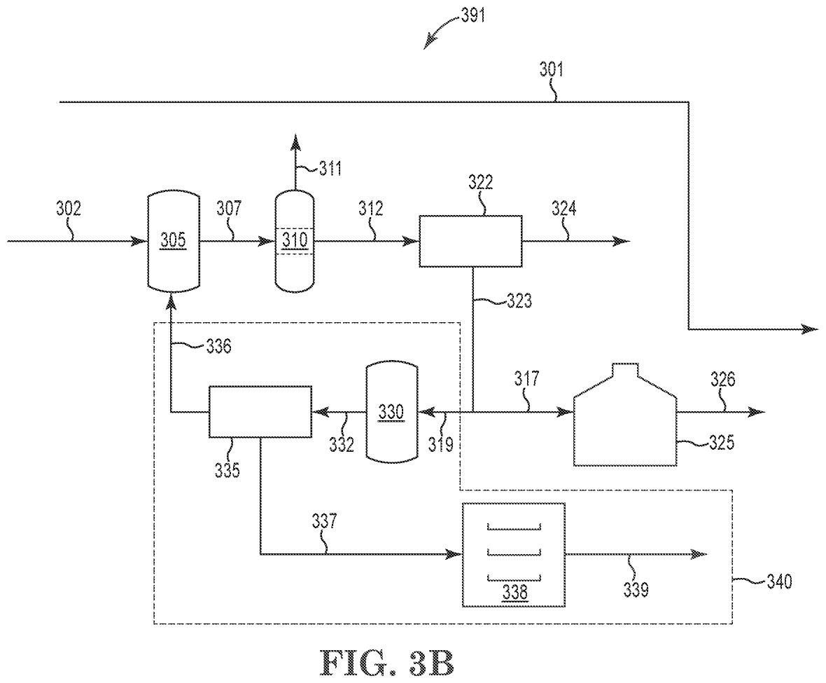

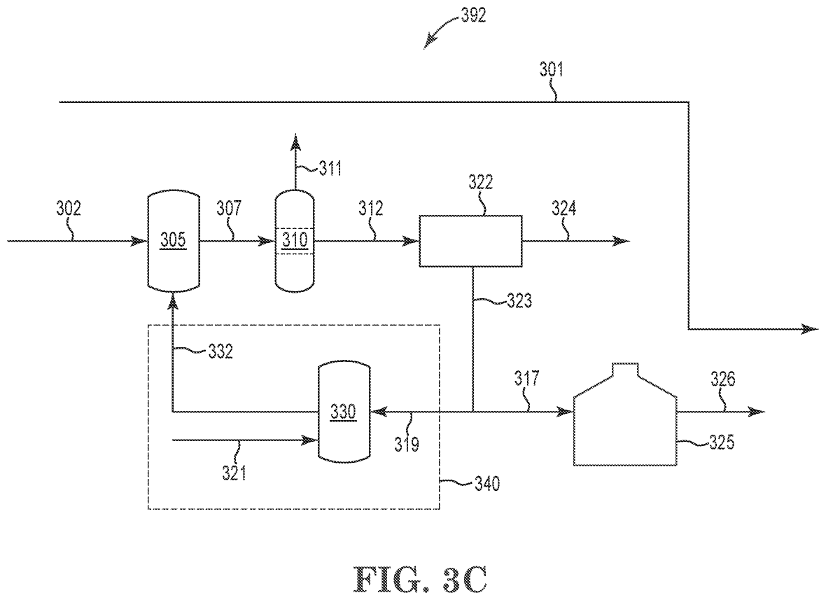

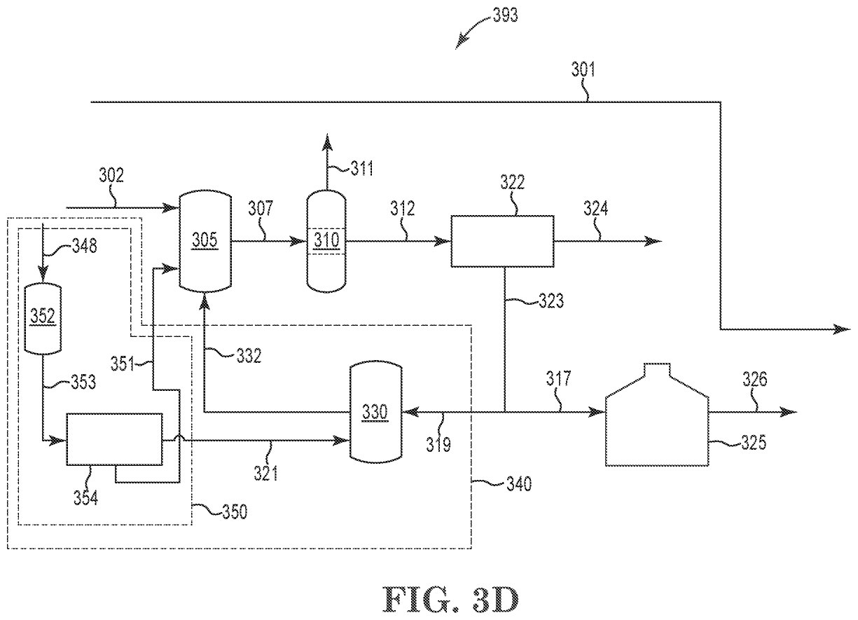

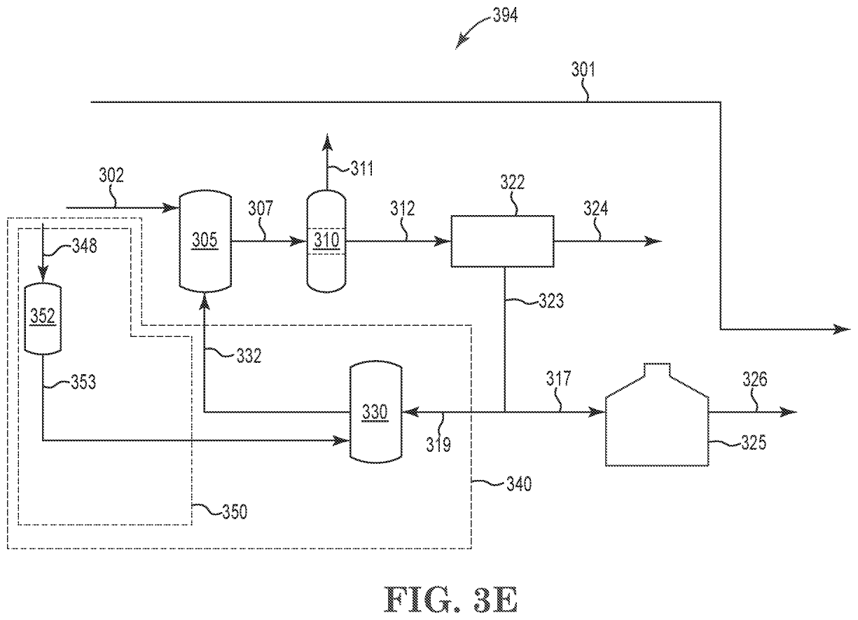

Reference is now made to non-limiting examples shown in each of FIGS. 2-3F to illustrate aspects of the present disclosure in the context of a bioprocessing facility for converting corn into a biochemical utilizing one or more enzymes and a fermentative microorganism. FIG. 2 depicts an illustrative example of a bioprocessing facility 200 having a primary process flow 201 and a secondary process flow 240. Primary process flow 201 includes a primary fermenter 205 that is in fluid communication with one or more sources 202 of material (e.g., corn flour, make-up water, enzymes, etc.) used to provide a fermentable composition that can ferment in primary fermenter 205. The enzymes hydrolyze components of the corn to allow a fermentative microorganism, e.g., yeast, to covert one or more products of hydrolysis, e.g., glucose, to a target biochemical, e.g., ethanol, and form a broth or beer 207.

The primary process flow also includes a separation system 210 (e.g., filtration, precipitation, distillation, and the like) that is in fluid communication with the primary fermenter 205 to receive the beer stream 207, which is separated into target biochemical stream 211 (e.g., ethanol) and whole stillage stream 212 as a co-product. A portion 217 of the whole stillage stream 212 is separated in the primary process flow 201 by a solid-liquid separation 222 (e.g., centrifugation), into a solids portion 224 having more solids (e.g., cake or retentate referred to as wet cake) and a liquid portion 223 having less solids (e.g., centrate or filtrate referred to as thin stillage). The thin stillage 223 is exposed to an evaporator system 225 to form a syrup 226. Corn oil can be separated from the syrup 226 if desired. The wet cake 224 can be dried if a dryer if desired. In some embodiments, syrup 226 can be combined with wet cake 224 prior to drying in a dryer system.

Referring to the secondary process flow 240, a portion 218 of the whole stillage stream 212 is transported to a secondary fermentation system 230 (enzyme production system) and used as growth medium for an enzyme producing microorganism, e.g. yeast or filamentous fungi. The enzyme produced may be one or more useful enzymes such as protease, lipase, esterase, phytase, amylase, cellulase, hemicellulase, xylanase, or pectinase. Optionally, a whole broth 232 from the secondary fermentation system 230 is separated in a separation system 235 (e.g., filtration, centrifugation, and the like) into an enzyme-containing thin stillage portion 236 and a wet cake portion 237. At least a portion of the enzyme-containing thin stillage portion 236 is recycled as enzyme containing backset to the primary fermenter 205 to provide enzyme for hydrolyzing corn components. In some embodiments, the separation of the broth 232 can be carried out so that the solids portion portion 237 can include more microorganisms from secondary fermentation 230 as compared to the enzyme-containing thin stillage portion 236, and the enzyme-containing thin stillage portion 236 can include more enzymes as compared to the solids portion 237. Conducting the separation to reduce the number of microorganisms present in the enzyme-containing thin stillage portion 236 can be advantageous in some embodiments. For example, enzyme producing microorganisms from secondary fermentation 230 may produce one or more biochemicals which are inhibitory to the microorganisms selected for primary fermentation if the microorganisms from secondary fermentation 230 are provided to the primary fermentation 205. For example, an enzyme producing bacteria from secondary fermentation 230 may produce enzymes during aerobic fermentation in secondary fermentation 230, but may produce one or more organic acids if the bacteria are viable and present during an anaerobic fermentation in primary fermenter 205. One or more of the organic acids may be inhibitory to a microorganism such as yeast present in the primary fermenter 205.

The solids portion 237 may be dried in a dryer system and sold as an animal feed and/or combined with wet cake stream 224.

FIG. 3A depicts an illustrative example of a bioprocessing facility 300 having a primary process flow 301 and a secondary process flow 340. FIG. 3A is similar to FIG. 2, but uses a portion of thin stillage instead of whole stillage as feedstock for secondary fermentation 330. Also, the secondary process flow 340 shows a solids portion 337 separated from whole broth 332 via a solid-liquid separation 322 (e.g., centrifugation) and has enough moisture that at least a portion of the solids portion 337 is introduced into an evaporator in the primary process flow 301. Although not shown, in some embodiments at least a portion of (or all of) solids portion 337 may be introduced into the primary process flow (e.g., combined with wet cake 324).

Primary process flow 301 includes a primary fermenter 305 that is in fluid communication with one or more sources 302 of material (e.g., corn flour, make-up water, enzymes, etc.) used to provide a fermentable composition that can ferment in primary fermenter 302. The enzymes hydrolyze components of the corn to allow a fermentative microorganism, e.g., yeast, to covert one or more products of hydrolysis, e.g., glucose, to a target biochemical, e.g., ethanol, and form a broth or beer 307.

The primary process flow also includes a separation system 310 (e.g., filtration, precipitation, distillation, and the like) that is in fluid communication with the primary fermenter 305 to receive the beer stream 307, which is separated into target biochemical stream 311 (e.g., ethanol) and whole stillage stream 312 as a co-product. The whole stillage stream 312 is separated in the primary process flow 201 by a solid-liquid separation 322 (e.g., centrifugation), into a solids portion 324 having more solids (e.g., cake or retentate referred to as wet cake) and a liquid portion 323 having less solids (e.g., centrate or filtrate referred to as thin stillage). A portion 317 of the thin stillage 323 is exposed to an evaporator system 325 to form a syrup 326. Corn oil can be separated from the syrup 326 if desired. The wet cake 324 can be dried if a dryer if desired. In some embodiments, syrup 326 can be combined with wet cake 324 prior to drying in a dryer system.

Referring to the secondary process flow 340, a portion 319 of the thin stillage stream 323 is transported to a secondary fermentation system 330 (enzyme production system) and used as growth medium for an enzyme producing microorganism, e.g. yeast or filamentous fungi. The enzyme produced may be one or more useful enzymes such as protease, lipase, esterase, phytase, amylase, cellulase, hemicellulase, xylanase, or pectinase. Optionally, the broth 332 from the secondary fermentation is separated in a solid-liquid separation 322 (e.g., centrifugation) into an enzyme-containing thin stillage portion 336 and a solids portion 337. At least a portion of the enzyme-containing thin stillage portion 336 is recycled as enzyme containing backset to the primary fermenter 305 to provide enzyme for hydrolyzing corn components. Although not shown, in some embodiments not all of enzyme-containing thin stillage portion 336 is needed in primary fermentation system 305 so for water-balance purposes at the bioprocessing facility 300 at least a portion of the enzyme-containing thin stillage portion 336 may be introduced to evaporator system 325. In some embodiments, the solids portion 337 can include more microorganisms from secondary fermentation 330 as compared to the enzyme-containing thin stillage portion 336, and the enzyme-containing thin stillage portion 336 can include more enzymes as compared to the solids portion 337. Reducing the amount of microorganisms present in the enzyme-containing thin stillage portion 336 can be advantageous in some embodiments. For example, enzyme producing microorganisms from secondary fermentation 330 may produce one or more biochemicals, e.g., if the microorganisms are present in primary fermenter 305, which are inhibitory to the microorganisms selected for primary fermentation. For example, an enzyme producing bacteria from secondary fermentation 330 may produce enzymes during aerobic fermentation in secondary fermentation 330, but may produce one or more organic acids if the bacteria are viable and present during an anaerobic fermentation in primary fermenter 305. One or more of the organic acids may be inhibitory to a microorganism such as yeast present in the primary fermenter 305.

FIG. 3B depicts an illustrative example of a bioprocessing facility 391 having a primary process flow 301 and a secondary process flow 340. Bioprocessing facility 391 in FIG. 3B is substantially the same as bioprocessing facility 300 in FIG. 3A, and the same reference characters are used to illustrate like features among FIGS. 3B an 3A. FIG. 3B differs from FIG. 3A in that the solids portion 337 in FIG. 3B is dried in a dryer system 338 to form a dried, high protein feed 339.

FIG. 3C depicts an illustrative example of a bioprocessing facility 392 having a primary process flow 301 and a secondary process flow 340. Bioprocessing facility 392 in FIG. 3C is substantially the same as bioprocessing facility 300 in FIG. 3A, and the same reference characters are used to illustrate like features among FIGS. 3C an 3A. FIG. 3C differs from FIG. 3A in that the broth 332 from secondary fermenter 330 is sent as a whole broth backset to primary fermenter 305 instead of first being separated. A whole broth backset 332 can be provided to primary fermenter 305 without first separating enzymes from broth 332 for one or more reasons, as desired. For example, the enzyme-producing microorganisms from secondary fermentation 330 may not be inhibitory to microorganisms used in primary fermentation 305. For example, enzyme-producing microorganisms from secondary fermentation 330 may not produce inhibitory compounds if the microorganisms are present during primary fermentation. As another example, enzyme-producing microorganisms from secondary fermentation 330 may not be active in primary fermentation 305 and, therefore, may not compete for nutrient sources and/or carbon sources. Enzyme-producing microorganisms from secondary fermentation 330 may not be active in primary fermentation 305 for a variety of reasons such as being optionally exposed to heat, a lysis system, etc.(not shown).

Also, FIG. 3C shows a supplemental feedstock 321 being provided to secondary fermentation 330. Supplemental feedstock 321 can be provided in a variety of forms and from a variety of sources. For example, supplemental feedstock 321 can include flour (e.g., corn flour) and/or monomeric sugar that is directly fed into secondary fermenter 330. If the supplemental feedstock 321 includes corn flour, the corn starch can be hydrolyzed into monomeric sugars during secondary fermentation 330 via SSF. The corn flour 321 can be prepared into corn flour from corn grain by a feedstock system present in bioprocessing facility 392 (e.g., a feedstock system used for primary fermentation 305) and/or corn flour 321 can be prepared at a facility external to bioprocessing facility 392 (e.g., at an off-site milling facility). Similarly, monomeric sugars can be provided directly to secondary fermentation via one or more sub-systems within bioprocessing facility 392 and/or can be prepared at a facility external to bioprocessing facility 392 (e.g., at an off-site sugar facility). Several non-limiting examples of providing supplemental feedstock 321 using a sub-system within the bioprocessing facility are illustrated below with respect to FIGS. 3D and 3E.

FIG. 3D depicts an illustrative example of a bioprocessing facility 393 having a primary process flow 301 and a secondary process flow 340. Bioprocessing facility 393 in FIG. 3D is substantially the same as bioprocessing facility 392 in FIG. 3C, and the same reference characters are used to illustrate like features among FIGS. 3D and 3C. FIG. 3D differs from FIG. 3C in that bioprocessing facility 393 illustrates an embodiment of providing supplemental feedstock 321. As shown in FIG. 3D, bioprocessing facility 393 includes a feedstock system 350 that forms a sugar stream 321 that is fed to secondary fermenter 330 as supplemental feedstock and used by one or more microorganisms to make enzyme. Feedstock system 350 can receive a raw feedstock 348 and process it to form a sugar or starch stream 321, and a “rejects” stream 351. Raw feedstock 348 may be the same or different from the feedstock used in a feedstock system for primary fermentation system 305. As shown in FIG. 3D, at least a portion sugar or starch stream 321 is provided to secondary fermentation system 330 as supplemental feedstock. As shown in FIG. 3D, starch present in feedstock 348 (e.g., grain mash) can be hydrolyzed in tank 352 to form a hydrolysate 353. The hydrolysate can be separated by a separation system 354 into the sugar stream 321 and rejects stream 351.

Separation system 354 can include one or more centrifuges (e.g., two-phase vertical disk stack centrifuge, three-phase vertical disk stack centrifuge, filtration centrifuge), one or more decanters (e.g., filtration decanters), one or more filters (e.g., fiber filter, rotary vacuum drum filter, filter device having one or more membrane filters), one or more screens (e.g., a “DSM” screen, which refers to a Dutch State Mines screen or sieve bend screen, and is a curved concave wedge bar type of stationary screen; a pressure screen; paddle screen; rotary drum screen; centrifugal screener; linear motion screen; vacu-deck screen; etc.), one or more brush strainers, one or more vibratory separators, one or more hydrocyclones, one or more presses, combinations of these and the like, to separate an aqueous enzyme composition (e.g., a clarified broth) from solids in a broth to concentrate the enzymes. Multiple separation systems can be used together and arranged in a parallel and/or series configuration. Depending on the separation system selected, one or more process input streams, including hydrolysate, can be separated into two or more output streams including at least sugar stream 321 and at least one rejects stream 351.

FIG. 3E depicts an illustrative example of a bioprocessing facility 394 having a primary process flow 301 and a secondary process flow 340. Bioprocessing facility 394 in FIG. 3E is substantially the same as bioprocessing facility 393 in FIG. 3D, and the same reference characters are used to illustrate like features among FIGS. 3D and 3E. FIG. 3E differs from FIG. 3D in that feedstock system 350 does not include a separation system 354. The hydrolysate formed in tank 352 is fed directly to secondary fermenter 330.

### Example 1

Example 1 was a bench-top experiment that evaluated, referring to FIG. 1A, using whole broth 132 from a secondary fermentation 130 as backset in a “pre-blend” for primary fermentation system 105.

Enzyme Broth Production—“Secondary Fermentation”

6.6 L bioreactors were filled with 2000 mL of commercial thin stillage from a raw starch hydrolysis dry grind ethanol plant. The reactors and thin stillage were then heated to and held at 70° C. for a minimum of 2 hours to pasteurize both the thin stillage and bioreactor. After pasteurization, the reactor contents were cooled and subsequently temperature controlled at 35° C. or 40° C. A calibrated pH probe and DO probe were then inserted into each reactor. The reactor material was then pH adjusted to pH 7 using 30% ammonium hydroxide and the bioreactor automatic controls; pH control was set at 7 until harvest. Next, DO probes were calibrated at 500 RPM and 0.5 SLPM using nitrogen for 0% and air for 100%. Aeration and mixing would remain at 500 RPM and 0.5 SLPM air for the remainder of the growth/production. At the same time and signifying the start of the aerobic fermentation, 1000 ppm urea, commercial enzyme cocktails (alpha-amylase, gluco-amylase, cellulases, and xylanases), and alpha-amylase producing Bacillus subtilis crème aerobically propagated using a yeast extract, peptone, and glucose medium for an ˜0.1 g dry cell weight/L loading were added to the reactor. Aerobic fermentation was performed for ˜16 h. After 16 h, the broth was harvested and frozen for future use.

### Yeast Propagation for “Primary Fermentation”

A lab scale anaerobic propagation was performed in a 125 mL glass bottle with vented cap. To start, corn flour (from commercial facility) and pre-blend (from commercial facility) were added. The mixture was then mixed and pH adjusted to 4.6 using sulfuric acid. To start the fermentation, commercial antibiotic, commercial alpha-amylase, commercial gluco-amylase, urea, and commercial yeast crème were added to the bottle followed by incubation in a shaking water batch at 150 RPM and 32.2° C. The yeast was allowed to propagate for ˜8 h.

### Raw Starch Hydrolysis and “Primary Fermentation”

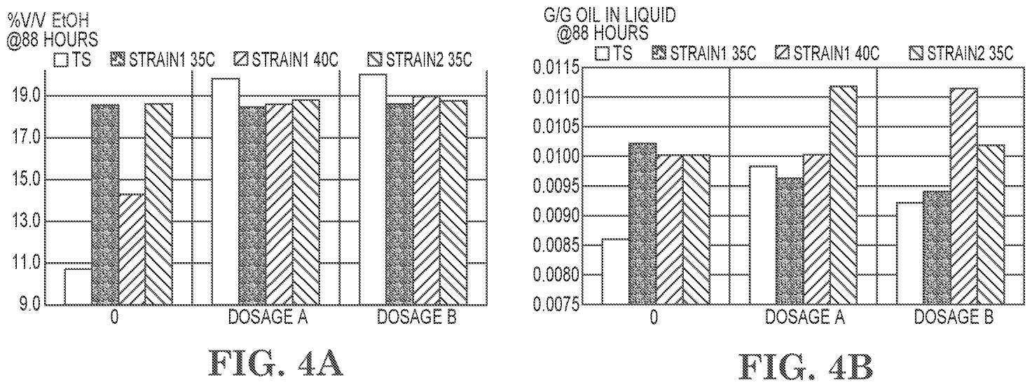

Lab scale anaerobic fermentations were performed in 125 mL glass bottles with vented caps. To start, corn flour (from commercial facility), thin stillage (from commercial facility) or enzyme broth (produced using above method), and RO water were added to each bottle. Thin stillage/enzyme broth was added at a 50:50 ratio to mimic commercial plant operations. Next, the bottle contents were mixed and pH adjusted to 4.6 using sulfuric acid. Prior to the start of fermentation, commercial anti-biotics, gluco-amylase, and urea were added at commercially relevant doses. Finally, the above described yeast propagation was dosed at a commercially relevant dose before placing the fermentation bottles in a water bath at 30-32° C. for ˜88 h. TS designates the use of commercial thin stillage, Strain 1 35 C designates an Bacillus subtilis alpha-amylase producing strain 1 enzyme broth produced at 35° C. and pH 7, Strain 1 40 C designates the same Bacillus subtilis alpha-amylase producing strain 1 enzyme broth produced at 40° C. & pH 7, and Strain 2 35 C designates a different Bacillus subtilis alpha-amylase producing strain 2 enzyme broth produced at 35° C. & pH 7. Referring to the x-axis in each of FIGS. 4A and 4B, “0” means that no exogeneous alpha-amylase was added to any of the samples. The only alpha amylase present would be that present in an enzyme broth, pre-blend, and corn. Dosage A refers to a dosage of alpha-amylase exogenously added to TS and a corresponding dosage produced in each enzyme broth. Dosage B refers to a dosage of alpha-amylase exogenously added to TS and a corresponding dosage produced in each enzyme broth, where the Dosage B was double the dosage of Dosage A.

### Example 2

Example 2 was a bench-top experiment that evaluated, referring to FIG. 1A, using whole broth 132 from a secondary fermentation 130 to supplement a “pre-blend” for primary fermentation system 105 with an “accessory enzyme” such as xylanase.

Enzyme Broth Production—“Secondary Fermentation”

6.6 L bioreactors were filled with 2000 mL of commercial thin stillage from a raw starch hydrolysis dry grind ethanol plant. The reactors and thin stillage were then heated to and held at 70° C. for a minimum of 2 h to pasteurize both the thin stillage and bioreactor. After pasteurization, the reactor contents were cooled and subsequently temperature controlled at 35° C. or 40° C. A calibrated pH probe and DO probe were then inserted into each reactor. The reactor material was then pH adjusted to either pH 5.5 or 7 using 30% ammonium hydroxide and the bioreactor automatic controls; pH control was set at the respective initial pH until harvest. Next, DO probes were calibrated at 500 RPM and 0.5 SLPM using nitrogen for 0% and air for 100%. Aeration and mixing would remain at 500 RPM and 0.5 SLPM air for the remainder of the growth/production. At the same time and signifying the start of the aerobic fermentation, 1000 ppm urea, commercial enzyme cocktails (alpha-amylase, gluco-amylase, and cellulases), and xylanase producing Bacillus subtilis crème aerobically propagated using a yeast extract, peptone, and glucose medium for an ˜0.1 g dry cell weight/L loading were added to the reactor. Aerobic fermentation was performed for ˜16 h. After 16 h, the broth was harvested and frozen for future use.

### Yeast Propagation for “Primary Fermentation”

A lab scale anaerobic propagation was performed in a 125 mL glass bottle with vented cap. To start, corn flour (from commercial facility) and pre-blend (from commercial facility) were added. The mixture was then mixed and pH adjusted to 4.6 using sulfuric acid. To start the fermentation, commercial antibiotic, commercial alpha-amylase, commercial gluco-amylase, urea, and commercial yeast crème were added to the bottle followed by incubation in a shaking water batch at 150 RPM and 32.2° C. The yeast was allowed to propagate for ˜8 h.

### Raw Starch Hydrolysis and “Primary Fermentation”

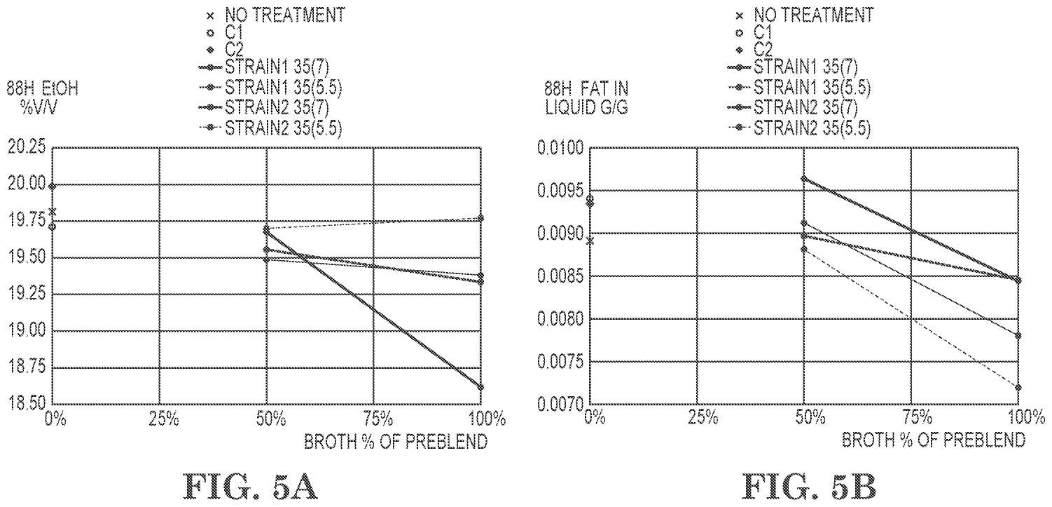

Lab scale anaerobic fermentations were performed in 125 mL glass bottles with vented caps. To start, corn flour (from commercial facility), thin stillage (from commercial facility) or enzyme broth (produced using above method), and RO water were added to each bottle. Thin stillage/enzyme broth was added at a 50:50 ratio to mimic commercial plant operations; the thin stillage/enzyme broth also varied at 100% thin stillage, 50% thin stillage:50% enzyme broth, and 100% enzyme broth. Next, the bottle contents were mixed and pH adjusted to 4.6 using sulfuric acid. Prior to the start of fermentation, commercial anti-biotics, gluco-amylase, alpha-amylase and urea were added at commercially relevant doses. Referring to FIGS. 5A and 5B, two different commercial xylanase cocktails (“C1” and “C2”) were added to two separate 0% enzyme broth (100% thin stillage) fermentation bottles; no added xylanase was added to a 0% enzyme broth control (“No Treatment”) and to fermentations using 50% or 100% enzyme broth. Finally, the above-described yeast propagation was dosed at a commercially relevant dose before placing the fermentation bottles in a water bath at 30-32° C. for ˜88 h. Strain1 35(7) designates Bacillus subtilis xylanase producing strain 1 enzyme broth produced at 35° C. and pH 7, Strain1 35(5.5) designates Bacillus subtilis xylanase producing strain 1 enzyme broth produced at 35° C. and pH 5.5, Strain2 35(7) designates Bacillus subtilis xylanase producing strain 2 enzyme broth produced at 35° C. and pH 7, and Strain1 35(5.5) designates Bacillus subtilis xylanase producing strain 2 enzyme broth produced at 35° C. and pH 5.5.

### Example 3

Example 3 was a bench-top experiment that evaluated, referring to FIG. 1A, increasing corn feedstock added to primary fermentation, and separating whole broth 132 from secondary fermentation 130 at 135 to separate enzyme from microorganism (Bacillus) and remove potential inhibition by Bacillus in primary fermentation system 105.

Enzyme Broth Production—“Secondary Fermentation”

6.6 L bioreactors were filled with 2000 mL of commercial thin stillage from a raw starch hydrolysis dry grind ethanol plant. The reactors and thin stillage were then heated to and held at 70° C. for a minimum of 2 h to pasteurize both the thin stillage and bioreactor. After pasteurization, the reactor contents were cooled and subsequently temperature controlled at 35° C. A calibrated pH probe and DO probe were then inserted into each reactor. The reactor material was then pH adjusted to pH 7 using 30% ammonium hydroxide and the bioreactor automatic controls; pH control was set at 7 until harvest. Next, DO probes were calibrated at 500 RPM and 0.5 SLPM using nitrogen for 0% and air for 100%. Aeration and mixing would remain at 500 RPM and 0.5 SLPM air for the remainder of the growth/production. At the same time and signifying the start of the aerobic fermentation, 1000 ppm urea, commercial enzyme cocktails (alpha-amylase, gluco-amylase, and cellulases), and xylanase producing Bacillus subtilis crème aerobically propagated using a yeast extract, peptone, and glucose medium for an ˜0.1 g dry cell weight/L loading were added to the reactor. Aerobic fermentation was performed for ˜16 h. After 16 h, the broth was harvested and frozen or further processed as described below. The xylanase producing Bacillus subtilis for this example is the same as the Bacillus subtilis xylanase producing strain 2 in Example 2 above.

Enzyme Broth Post-Production Separation

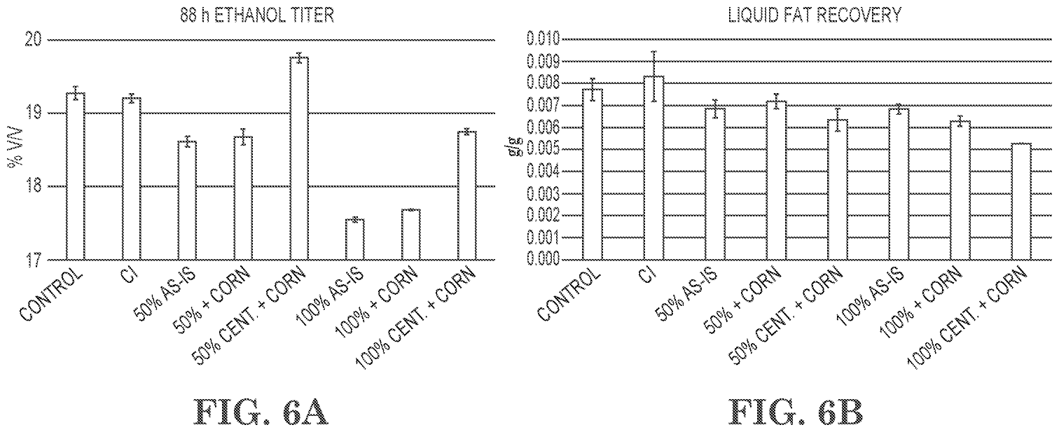

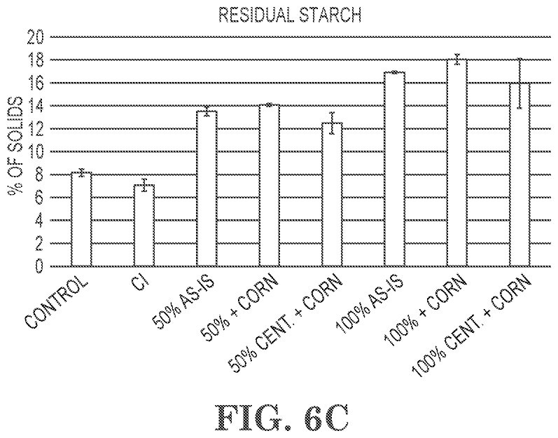

A portion of whole enzyme broth was added to 1 L centrifuge bottles and centrifuged at 4200 RPM for 10 minutes. The resulting supernatant was poured off, collected, and frozen. It was observed that the enzyme broth and supernatant were not homogeneous, and that oil phase tended to stick to the centrifuge tube, thereby making the data in FIG. 6B difficult to interpret. While not being bound by theory, it is believed that Bacillus subtilis that was used produced a variety of non-target enzymes (as compared to “target” enzymes that are overexpressed for industrial production) that broke down one or more of protein, carbohydrate, fiber, etc. and liberated a relatively high level of oil during secondary fermentation. Also, while not being bound by theory, it is believed that in some embodiments, using a different microorganism such as yeast for secondary fermentation may improve solid-liquid separation because a yeast can be selected to not produce as many non-target enzymes and, therefore, may not liberate as much oil from the feedstock in secondary fermentation.

Yeast Propagation for “Primary Fermentation”

A lab scale anaerobic propagation was performed in a 125 mL glass bottle with vented cap. To start, corn flour (from commercial facility) and pre-blend (from commercial facility) were added. The mixture was then mixed and pH adjusted to 4.6 using sulfuric acid. To start the fermentation, commercial antibiotic, commercial alpha-amylase, commercial gluco-amylase, urea, and commercial yeast crème were added to the bottle followed by incubation in a shaking water batch at 150 RPM and 32.2° C. The yeast was allowed to propagate for ˜8 h.

Raw Starch Hydrolysis and “Primary Fermentation”

Lab scale anaerobic fermentations were performed in 125 mL glass bottles with vented caps. To start, corn flour (from commercial facility), thin stillage (from commercial facility) or enzyme broth (produced using above method), and RO water were added to each bottle. Corn flour additions were made at different levels. Select fermentations included additional corn was added to make up for solids loss during enzyme production and during centrifugation; corn was added to match the total solids in the control fermentation. The “Control” was 100% thin stillage with no added xylanase. Thin stillage and enzyme broth were combined at a 50:50 ratio and then combined with 50% RO water (25% thin stillage; 25% enzyme broth; and 50% RO water) to mimic commercial plant operations and is designated by “50%” in FIGS. 6A-6C. Samples using 100% enzyme broth and no thin stillage were combined with 50% RO water (50% enzyme broth and 50% RO water), and are designated with a “100%” in FIGS. 6A-6C. Next, the bottle contents were mixed and pH adjusted to 4.6 using sulfuric acid. Prior to the start of fermentation, commercial anti-biotics, gluco-amylase, alpha-amylase and urea were added at commercially relevant doses. Referring to FIGS. 6A, 6B, and 6C, a commercial xylanase (“C1”) was added to a 0% enzyme broth (100% thin stillage) fermentation; no commercial xylanase was added to fermentations using 50% or 100% enzyme broth. Also, “As-Is” means a whole enzyme broth was used and was not separated; “+Corn” means additional corn was added (see above) to As-Is enzyme broth; and “Cent.+Corn” means enzyme broth supernatant was used and that additional corn was added (see above). Finally, the above described yeast propagation was dosed at a commercially relevant dose before placing the fermentation bottles in a water bath at 30-32° C. for ˜88 h. All enzyme broth produced for this experiment used the same xylanase producing Bacillus subtilis strain.

Referring to FIGS. 6A and 6C, it can be seen that separating microorganisms from the whole broth and using the supernatant along with additional corn improved the ethanol production and reduced the residual starch.

What is claimed is:

1. A bioprocessing facility having enzyme production, wherein the bioprocessing facility comprises:
a primary fermentation system configured to expose fermentable composition to fermentation conditions and produce beer comprising one or more bioproducts;
a separation system in fluid communication with the primary fermentation system, wherein the separation system is configured to separate at least a portion of at least one of the one or more bioproducts from beer produced in the primary fermentation system and form whole stillage;
a secondary fermentation system in fluid communication with one or more sources of a stillage composition, wherein the one or more sources of a stillage composition are chosen from whole stillage, stillage derived from whole stillage, and combinations thereof, wherein the secondary fermentation system is configured to form fermentable composition using at least a portion of the one or more sources of a stillage composition and one or more microorganisms, wherein the secondary fermentation system is configured to expose fermentable composition to fermentation conditions so the one or more microorganisms can produce one or more bioproducts and form secondary fermentation broth, wherein the one or more bioproducts comprise one or more enzymes, and wherein the secondary fermentation system is in fluid communication with one or more systems in the bioprocessing facility so the one or more systems can combine at least a portion of the one or more enzymes with one or more process compositions in the bioprocessing facility to hydrolyze at least one substrate in the one or more process compositions.

2. The bioprocessing facility of claim 1, wherein the separation system is a first separation system comprising a distillation system configured to separate at least a portion of at least one of the one or more bioproducts from beer produced in the primary fermentation system and form whole stillage, and further comprising a second separation system in fluid communication with secondary fermentation broth produced in the secondary fermentation system, wherein the secondary fermentation system is configured to separate at least a portion of the one or more enzymes from secondary fermentation broth produced in the secondary fermentation system.

3. The bioprocessing facility of claim 2, wherein the second separation system comprises one or more centrifuges, one or more decanters, one or more filters, one or more screens, one or more brush strainers, one or more vibratory separators, one or more hydrocyclones, one or more presses, and combinations thereof.

4. The bioprocessing facility of claim 1, wherein the primary fermentation system is configured to expose fermentable composition to anaerobic fermentation conditions to produce beer and the secondary fermentation system is configured to expose fermentable composition to aerobic fermentation conditions to produce secondary fermentation broth.

5. The bioprocessing facility of claim 1, wherein at least a portion of the one or more enzymes produced in the secondary fermentation system is in fluid communication with the primary fermentation system, wherein the one or more enzymes hydrolyze polysaccharide.

6. The bioprocessing facility of claim 1, wherein the one or more sources of a stillage composition are chosen from whole stillage, thin stillage, syrup, and combinations thereof.

7. The bioprocessing facility of claim 1, wherein the one or more sources of a stillage composition comprise one or more defatted stillage compositions.

8. The bioprocessing facility of claim 1, wherein the primary fermentation system is in fluid communication with a feedstock system to receive feedstock and form fermentable composition.

9. The bioprocessing facility of claim 8, wherein the primary fermentation system is in fluid communication with a first feedstock system, wherein the secondary fermentation system is in fluid communication with a second feedstock system to receive feedstock and form fermentable composition, wherein the first feedstock system and the second feedstock system are the same feedstock system or are different feedstock systems.

10. The bioprocessing facility of claim 9, wherein the first feedstock system and the second feedstock system are different feedstock systems, and wherein the second feedstock system is configured to form at least one stream from grain feedstock, wherein the at least one stream comprises sugar and/or starch, and wherein the at least one stream is in fluid communication with the secondary fermentation system to form fermentable composition.

11. The bioprocessing facility of claim 10, wherein the second feedstock system is configured to process grain feedstock to form at least a first stream comprising the sugar and/or the starch, and a second stream comprising residual grain feedstock components, and wherein the bioprocessing facility is configured to combine at least a portion of the second stream with one or more process compositions in the bioprocessing facility.

12. The bioprocessing facility of claim 11, wherein the one or more process compositions are chosen from beer produced by the primary fermentation system, one or more stillage compositions, secondary fermentation broth produced by the secondary fermentation system, and combinations thereof.

13. A method of producing one or more enzymes in a bioprocessing facility, wherein the method comprises:
exposing fermentable composition to fermentation conditions in a primary fermentation system to produce beer comprising one or more bioproducts;
separating at least a portion of at least one of the one or more bioproducts from beer produced in the primary fermentation system to form whole stillage;
exposing fermentable composition to fermentation conditions in a secondary fermentation system so one or more microorganisms can produce one or more bioproducts, wherein the one or more bioproducts comprise one or more enzymes, wherein fermentable composition in the secondary fermentation system is formed from at least a portion of one or more stillage compositions produced in the bioprocessing facility, wherein the one or more stillage compositions are chosen from whole stillage, a stillage derived from whole stillage, and combinations thereof;
combining at least a portion of the one or more enzymes produced in the secondary fermentation system with one or more process compositions in the bioprocessing facility to hydrolyze at least one substrate in the one or more process compositions.

14. The method of claim 13, wherein separating at least a portion of at least one of the one or more bioproducts from beer produced in the primary fermentation system comprises distilling beer produced in the primary fermentation system to separate at least a portion of at least one of the one or more bioproducts from the beer and form whole stillage, and further comprising separating at least a portion of the one or more enzymes from secondary fermentation broth produced in the secondary fermentation system.

15. The method of claim 13, wherein fermentable composition in the primary fermentation system comprises one or more microorganisms that produce one or more alcohols under anaerobic fermentation conditions and wherein fermentable composition in the secondary fermentation system comprises one or more microorganisms that produce the one or more enzymes under aerobic fermentation conditions.

16. The method of claim 15, wherein fermentable composition in the primary fermentation system comprises at least one yeast that produces ethanol and wherein fermentable composition in the secondary fermentation system comprises at least one bacteria that produces one or more enzymes that hydrolyzes at least one substrate chosen from polysaccharide or protein.

17. The method of claim 16, wherein combining at least a portion of the one or more enzymes produced in the secondary fermentation system with one or more process compositions in the bioprocessing facility comprises combining the at least a portion of the one or more enzymes with one or more process compositions so that the at least a portion of the one or more enzymes are present in fermentable composition in the primary fermentation system.

18. The method of claim 13, further comprising:
providing feedstock derived from grain to the primary fermentation system to form fermentable composition; and
providing feedstock derived from grain to the secondary fermentation system to form fermentable composition.

19. The method of claim 18, wherein the feedstock provided to the primary fermentation system and the secondary fermentation system comprises corn flour.

20. The method of claim 18, further comprising a feedstock system configured to process grain feedstock to form at least a first stream comprising sugar and/or starch and a second stream comprising residual grain feedstock components, wherein the feedstock provided to the secondary fermentation system comprises the first stream, and wherein at least a portion of the second stream introduced into one or more of beer produced by first fermentation system, one or more stillage compositions produced in the bioprocessing facility, or secondary fermentation broth produced by secondary fermentation system.

21. The method of claim 15, wherein fermentable composition in the primary fermentation system comprises at least one yeast that produces a biochemical and wherein fermentable composition in the secondary fermentation system comprises at least one yeast that produces one or more enzymes that can hydrolyze at least one substrate chosen from polysaccharide or protein.
